(19) United States
(12) Patent Application Publication
(10) Pub. No.: US 2022/0226628 A1
(43) Pub. Date: Jul. 21, 2022

(54) Adapter to Facilitate Blood Draw and Infusion, Related Systems, And Methods

(71) Applicant: Becton, Dickinson and Company, Franklin Lakes, NJ (US)

(72) Inventor: S. Ray Isaacson, Layton, UT (US)

(21) Appl. No.: 17/579,118

(22) Filed: Jan. 19, 2022

Related U.S. Application Data
(60) Provisional application No. 63/139,621, filed on Jan. 20, 2021.

(51) Int. Cl. A61M 39/10; A61M 25/00; A61B 5/15; A61M 5/168; A61M 5/14

(52) U.S. Cl. CPC A61M 39/105; A61M 25/0097; A61B 5/150992; A61M 5/16813; A61M 2005/1403; A61B 5/150267; A61M 2039/1072; A61M 2039/1077; A61M 5/1413

(57) ABSTRACT

An adapter configured to couple to a catheter assembly. The adapter may include a first proximal port, a second proximal port, and a distal port. An infusion fluid pathway may extend between the first proximal port and the distal port. A blood withdrawal fluid pathway may extend between the second proximal port and the distal port. The adapter may include a one-way septum disposed in the first proximal port and configured to open to allow fluid flow in a distal direction at a pressure differential across the one-way septum. The one-way septum is disposed within the infusion fluid pathway. The adapter may include a two-way septum disposed in the second proximal port. The two-way septum may be disposed within the blood withdrawal fluid pathway.

### CROSS-REFERENCE TO RELATED APPLICATION

The present application claims priority to U.S. Provisional Application Ser. No. 63/139,621, entitled “Adapter to Facilitate Blood Draw and Infusion, Related Systems, and Methods”, filed Jan. 20, 2021, the entire disclosure of which is hereby incorporated by reference in its entirety.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient. Once collected, blood samples may be tested to determine the physiological and biochemical states of a patient, such as disease, mineral content, drug effectiveness, and organ function.

A common type of catheter is a peripheral intravenous catheter (“PIVC”) that is “over-the-needle.” As its name implies, the PIVC that is over-the-needle may be mounted over an introducer needle having a sharp distal tip. The PIVC and the introducer needle may be assembled so that the distal tip of the introducer needle extends beyond the distal tip of the PIVC with the bevel of the needle facing up away from skin of the patient. The PIVC and introducer needle are generally inserted at a shallow angle through the skin into the vasculature of the patient.

An integrated PIVC includes a PIVC coupled to an extension set. The extension set typically includes extension tubing that is integrated at one end into the catheter hub and that includes an adapter, such as a Y-adapter, coupled to the other end. The adapter may include one or more access ports (e.g., luer connectors).

Integrated PIVCs may be used to draw blood. In further detail, after insertion of the integrated PIVC into the patient's vasculature, blood may flow into the extension set and into the adapter. To facilitate this blood flow, a vent plug will typically be coupled to at least one of the access ports, which will allow air to escape the extension tube as the blood flows into the extension tube. A blood collection device may be coupled to the adapter. In response to blood filling the adapter, the clinician may begin collecting blood.

Blood draw through the PIVC presents various challenges. For example, although blood samples may be desired by the clinician at various points during a course of therapy, the PIVC may not be reliable for drawing blood after its initial insertion. Also, even just temporary use of the PIVC for blood withdrawal may prevent quick transitioning to infusion. In further detail, fluid is injected through the adapter to flush blood from the extension tubing following blood withdrawal. This process of flushing tends to be both time-consuming and inefficient as it is difficult to fully flush residual blood from within the adapter and access ports. The residual blood may increase a risk of bloodstream infection when a particular access port is for blood withdrawal and infusion of fluids into the patient's vasculature.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to devices, systems, and methods to facilitate automatic fluid infusion and blood withdrawal from catheters. Some embodiments in the present disclosure may provide different fluid pathways for withdrawing blood and for injecting fluids via an extension set. In some embodiments, an adapter may be configured to allow infusates to flow in a single direction through one of the different fluid pathways. In some embodiments, the adapter may be configured to allow blood to flow in two directions through another of the different fluid pathways. In this manner, some embodiments may enable fluids to be infused into a patient's vasculature with a reduced risk of blood contamination from a previous blood draw. Additionally, in some embodiments, blood may be automatically collected from the patient's vasculature for a diagnostic procedure, and then reinfused following the diagnostic procedure.

In some embodiments, a catheter system may include a catheter hub, which may include a distal end and a proximal end. In some embodiments, the catheter system may include a catheter, which may extend from the distal end of the catheter hub. In some embodiments, the catheter may include a PIVC, a peripherally-inserted central catheter, or a midline catheter. In some embodiments, the catheter system may include an adapter, which may be in fluid communication with the catheter hub and the catheter. In some embodiments, the adapter may include a first proximal port, a second proximal port, and a distal port. In some embodiments, an infusion fluid pathway may extend through the first proximal port and the distal port. In some embodiments, a blood withdrawal fluid pathway may extend through the second proximal port and the distal port.

In some embodiments, the catheter system may include a one-way septum, which may be disposed in the first proximal port and configured to open to allow fluid flow in a distal direction at a pressure differential across the one-way septum. In some embodiments, the one-way septum is disposed within the infusion fluid pathway. In some embodiments, the catheter system may include a two-way septum disposed in the second proximal port. In some embodiments, the two-way septum may be disposed within the blood withdrawal fluid pathway.

In some embodiments, the catheter system may include an extension tube. In some embodiments, the extension tube may include a distal end coupled to the catheter hub and a proximal end coupled to the distal port of the adapter. In some embodiments, the first proximal port may be aligned with the distal port and the infusion fluid pathway may be straight. In some embodiments, the second proximal port may include a side port disposed between the first proximal port and the distal port.

In some embodiments, the catheter system may include an infusion module. In some embodiments, the infusion module may include a first extension tube configured to direct a fluid in a distal direction through the infusion fluid pathway for infusion into a patient's vasculature. In some embodiments, the catheter system may include a diagnostic module. In some embodiments, the diagnostic module may include a second extension tube configured to direct blood in a proximal direction from the patient's vasculature through the blood withdrawal fluid pathway. In some embodiments, a distal end of the first extension tube may be coupled to the first proximal port and a distal end of the second extension tube is coupled to the second proximal port.

In some embodiments, the infusion module may include at least one of a pump and a gravity-based device to direct the fluid in the distal direction through the infusion fluid pathway. In some embodiments, the diagnostic module may be configured to apply a pressure differential across the two-way septum to open the two-way septum and direct blood in the proximal direction through the blood withdrawal fluid pathway. In some embodiments, the diagnostic module may include a pump to push a fluid in the distal direction through the blood withdrawal fluid pathway to reinfuse the blood into the patient's vasculature. In some embodiments, the diagnostic module may include at least one sensor to perform a diagnostic test on the blood.

In some embodiments, the catheter system may include a saline flush device. In some embodiments, the pump may be configured to reintroduce blood into the blood withdrawal pathway and distally through the catheter via the saline flush device. In some embodiments, the saline flush device may be embedded in the diagnostic module. In some embodiments, the saline flush device stands alone with respect to the diagnostic module.

In some embodiments, a method to facilitate fluid infusion and blood withdrawal may include priming the adapter. In some embodiments, the method may include directing a first fluid in a distal direction through the infusion pathway to infuse the first fluid into a patient's vasculature. In some embodiments, the method may include withdrawing blood from the patient's vasculature in a proximal direction through the blood withdrawal fluid pathway to direct the blood into a blood collection reservoir. In some embodiments, the method may include performing an analysis of the blood in the blood collection reservoir. In some embodiments, the method may include, after performing the analysis, pushing a second fluid in the distal direction through at least one of the blood collection reservoir and the blood withdrawal fluid pathway to reinfuse the blood into the patient's vasculature.

In some embodiments, directing the first fluid in the distal direction may include utilizing a flow of the first fluid to open a one-way septum disposed within the first proximal port. In some embodiments, in response to utilizing a flow of the first fluid to open the one-way septum, the two-way septum may remain closed. In some embodiments, in response to pushing the second fluid in the distal direction through at least one of the blood collection reservoir and the blood withdrawal fluid pathway to reinfuse the blood into the patient's vasculature, the one-way septum may remain closed.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1 illustrates an example catheter system to facilitate fluid infusion and blood withdrawal, according to some embodiments;

FIG. 2 illustrates the catheter system to facilitate fluid infusion and blood withdrawal, according to some embodiments;

FIG. 3 is a cross-sectional view of an adapter that includes a one-way septum and a two-way septum, according to some embodiments;

FIG. 4A is a cross-sectional view of the adapter of FIG. 3 illustrating an initial state where the adapter is primed, according to some embodiments;

FIG. 4B is a cross-sectional view of the adapter of FIG. 3 illustrating a blood withdrawal state, according to some embodiments;

FIG. 4C is a cross-sectional view of the adapter of FIG. 3 illustrating a blood reinfusion state, according to some embodiments; and

FIG. 4D is a cross-sectional view of the adapter of FIG. 3 illustrating an infusion state, according to some embodiments.

### DESCRIPTION OF EMBODIMENTS

Prior art catheter systems are not equipped for quick transitioning between blood withdrawal use and infusion use. Infusion or blood withdrawal through prior art adapters may result in mixing of fluids and the fluid going in a wrong direction for purposes of infusion or blood draw. Simply putting a needleless connector on each access port of an adapter may not solve the problem. Indeed, the needleless connector would create a fluid path that is open in both directions.

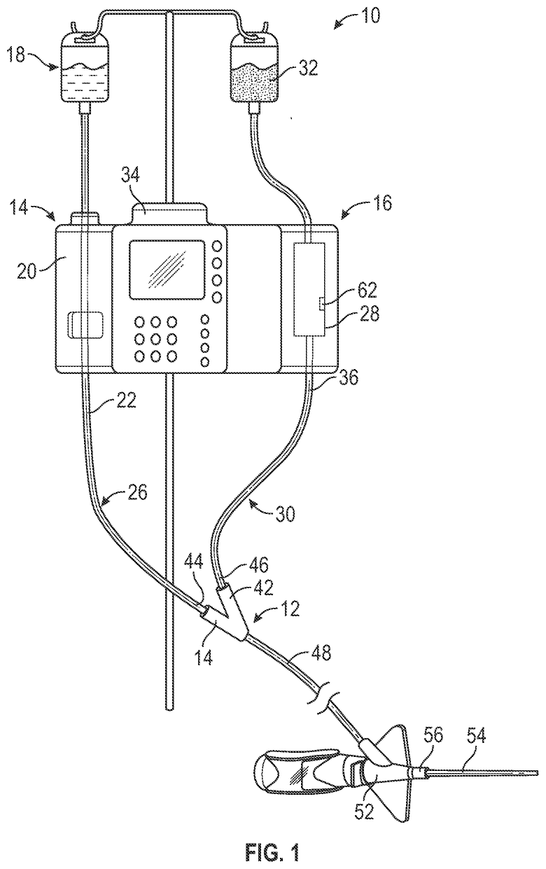

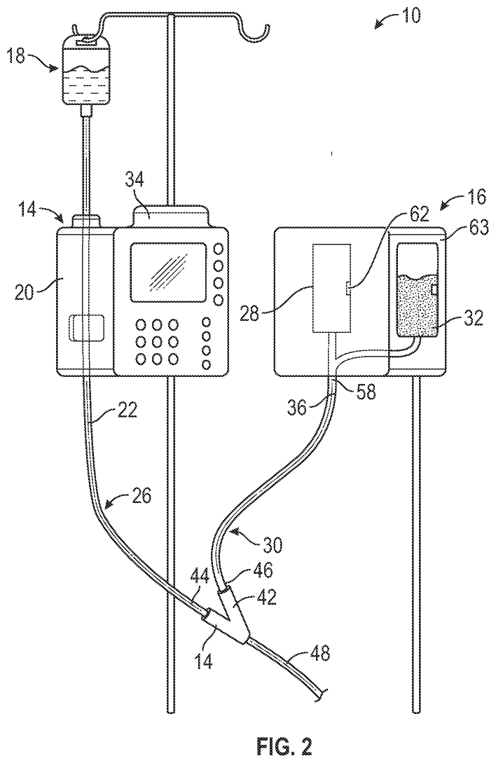

Referring now to FIGS. 1-2, a catheter system 10 in accordance with some embodiments may facilitate fluid infusion and blood withdrawal from a catheter in fluid communication with a patient's vasculature. Some embodiments of the catheter system 10 may provide a way to consistently and reliably provide infusion as well as blood withdrawal for diagnostic purposes. In some embodiments, in response to blood withdrawal and analysis of blood for diagnostic purposes, at least a portion of the blood may be returned to the patient's vasculature with a small amount of saline flush, and the line may be restored to its primary state for pump-based infusion.

In some embodiments, the catheter system 10 may include an adapter 12, such as, for example, a Y-adapter, to provide separate dedicated fluid pathways for withdrawing blood and injecting fluids into a patient's vasculature. For example, the adapter 12 may include septums configured to direct a flow of fluid or infusates in one direction (into a patient) through one pathway, and to direct blood to flow in either of two directions (out of the patient and/or into the patient) through another pathway. In this manner, some embodiments may enable the catheter to be used for both blood withdrawal and fluid infusion with a reduced risk of contamination from a prior blood draw.

As illustrated in FIG. 1, in some embodiments, the catheter system 10 may include an infusion module 14, a diagnostic module 16, and an adapter 12. In some embodiments, the infusion module 14 may receive infusates such as saline, medicaments, parenteral nutrition, and/or other suitable fluids from an infusate administration set 18. In some embodiments, the infusate administration set 18 may include an electronic infusion device, such as a pump 20, a gravity-based reservoir, or other suitable infusion device known to those in the art. Some embodiments of the infusion module 14 may include a first extension tube 22 to direct a fluid or infusate in a distal direction 24 through a first fluid pathway 26 for infusion into a patient's vasculature. The first fluid pathway 26 may be referred to in the present disclosure as “the infusion fluid pathway.” A distal end of the first extension tube 22 may be coupled to the adapter 12 by a press fit, for example, or by any other suitable method or device.

In some embodiments, the diagnostic module 16 may be positioned next to or in close proximity to the infusion module 14. In some embodiments, the diagnostic module 16 may include a blood collection reservoir 28 to collect blood withdrawn from the patient's vasculature through a second fluid pathway 30. The second fluid pathway 30 may be referred to in the present disclosure as “the blood withdrawal fluid pathway” and may also be used for reintroducing withdrawn blood into vasculature of the patient. In some embodiments, a saline flush device may include a saline flush reservoir 32, which may communicate with the diagnostic module 16 to facilitate reinfusion of the collected blood into the patient's vasculature, as discussed in more detail below.

In some embodiments, the saline flush reservoir 32 may hang above the diagnostic module 16 in a conventional set-up situation such that the saline or other fluid may be directed by gravity into the diagnostic module 16. In some embodiments, the diagnostic module 16 may be a stand-alone device that includes the saline flush reservoir 32. In these and other embodiments, a pump may apply positive pressure to the saline or other fluid to direct the fluid flow in the distal direction 24 through the second fluid pathway 30. In some embodiments, the diagnostic module 16 and the infusion module 14 may communicate with a shared control device 34 to control their respective fluid flows into and out of the patient's vasculature.

In some embodiments, the diagnostic module 16 may include a second extension tube 36 configured to direct blood in a proximal direction 38 away from the patient's vasculature through a second fluid pathway 30. In some embodiments, a distal end of the second extension tube 36 may be coupled to the adapter 12. In some embodiments, the saline flush reservoir 32 may also communicate with the second extension tube 36 to reinfuse the blood in a distal direction 24 into the patient's vasculature.

In some embodiments, the adapter 12 may include an infusion portion 40, which may include at least a portion of the first fluid pathway 26 and a blood withdrawal portion 42 including at least a portion of the second fluid pathway 30. In some embodiments, the distal end 44 of the first extension tube 22 may be coupled to the infusion portion 40 and the distal end 46 of the second extension tube 36 may be coupled to the blood withdrawal portion 42.

In some embodiments, the catheter system 10 may further include a third extension tube 48. In some embodiments, a distal end 50 of the third extension tube 48 may be coupled to a catheter hub 52. In some embodiments, the catheter hub 52 may include a catheter 54 extending from a distal end 56 thereof. In some embodiments, the catheter 54 may be in fluid communication with the patient's vasculature. In some embodiments, the catheter 74 may include a PIVC, a peripherally-inserted central catheter, or a midline catheter.

In some embodiments, the diagnostic module 16 may include a vacuum pump or other suitable device to apply a negative pressure to a proximal end 90 of the second extension tube 36. In response to activation of the vacuum pump or other such device, the diagnostic module 16 may direct blood in the proximal direction 38 through the second fluid pathway 30. In some embodiments, the diagnostic module 16 may be configured to collect blood from the patient's vasculature. In some embodiments, the blood may be collected into the blood collection reservoir 28. In some embodiments, the blood collection reservoir 28 may include or correspond to a testing compartment to collect and test the blood. Some embodiments of the testing compartment may include one or more sensors 62 to perform one or more of various diagnostic tests on the collected blood.

In some embodiments, the diagnostic module 16 may include a pump 63, which may be operatively coupled to the blood collection reservoir 28 and/or the saline flush reservoir 32 to push saline and/or blood in the distal direction 24 through the second fluid pathway 30. In some embodiments, the pump 20 may include a positive pressure pump. In some embodiments, in response to activation of the pump 20, saline or another suitable fluid may be forced from the saline flush reservoir 32 through the second fluid pathway 30. In this manner, some embodiments may utilize positive pressure applied to the saline or other fluid to thereby push blood in the distal direction 24 through the second fluid pathway 30 to reinfuse the blood into the patient's vasculature.

As illustrated in FIG. 1, in some embodiments, the infusion module 14 may be coupled to the diagnostic module 16. As illustrated in FIG. 1, in some embodiments, the saline flush reservoir may stand alone or be disposed in a container separate from the diagnostic module 16. In other embodiments, as illustrated in FIG. 2, the saline flush reservoir 32 may be embedded or disposed within the diagnostic module 16.

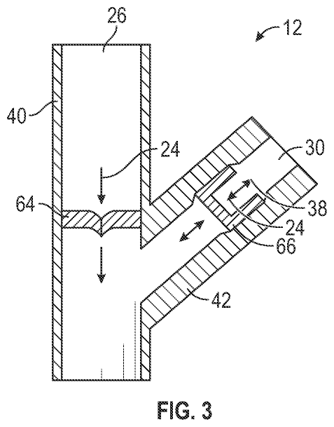

Referring now to FIG. 3, in some embodiments, a one-way septum 64 may be disposed within the infusion portion 40. In some embodiments, the one-way septum 64 may be a low-pressure one-way septum configured to automatically open in response to a flow of the fluid in the distal direction 24. In some embodiments, the one-way septum 64 may be configured to open in response to a small cracking pressure as applied by the pump 20 or gravity-based infusion, for example. In some embodiments, the one-way septum 64 may not be reversible; thus, application of a vacuum or a drop in applied pressure may close the one-way septum 64 to provide a fluid-tight seal.

In some embodiments, a two-way septum 66 may be disposed within the blood withdrawal portion 42. In some embodiments, the two-way septum 66 may include a passive septum configured to automatically open in response to a flow of the blood in the proximal direction 38 through the second fluid pathway 30.

In some embodiments, the two-way septum 66 may have uniform cracking pressures in both the proximal direction 38 and the distal direction 24. In some embodiments, the two-way septum 66 may be tuned to symmetric activation pressure in either the distal direction 24 or the proximal direction 38. In other embodiments, the two-way septum 66 may be tuned to asymmetric cracking pressures. For example, the two-way septum 66 may open in the proximal direction 38 for blood withdrawal in response to a pressure differential across the two-way septum, but the two-way septum may not open in the distal direction 24 for blood reinfusion in response to an opposite pressure differential of a same magnitude as the pressure differential.

In some embodiments, the two-way septum 66 may be strong enough to remain closed during pressure-based infusion through the infusion portion 40. Additionally, some embodiments of the two-way septum 66 may be sealed closed absent direct application of negative or positive pressure. In this manner, some embodiments of the two-way septum 66 may seal the blood withdrawal portion 42 such that any saline, drugs or other fluid infused through the first fluid pathway 26 may not reverse up the blood withdrawal portion 42 past the position of the two-way septum 66 and be inadvertently delivered to the patient.

During blood withdrawal or blood withdrawal, in some embodiments, a negative pressure or pumping force may be applied to the proximal end 90 of the second extension tube 36. In some embodiments, as mentioned above, this may trigger the one-way septum 64 to close and the two-way septum 66 to open, allowing for low-force blood withdrawal. In some embodiments, the negative pressure may trigger the two-way septum 66 to open such that the blood may travel in the proximal direction 38 up to the diagnostic module 16. Importantly, in some embodiments, the pressure applied to the proximal end 90 of the second extension tube 36 may be sufficiently gentle such that the blood may not hemolyze as it passes through the second fluid pathway 30.

In some embodiments, following blood withdrawal into the diagnostic module 16 and completion of the diagnostic test, the two-way septum 66 may allow blood to passively flow in the distal direction 24 through the two-way septum 66 to be reinfused into the patient's vasculature. In this manner, some embodiments of the catheter system 10 may reduce a chance of blood clotting or fouling after testing. In some embodiments, the blood may be reinfused by pushing a small amount of saline or other suitable fluid through the second fluid pathway 30 in the distal direction 24. In some embodiments, positive pressure applied by a pump, for example, may push the saline or other suitable fluid through the second fluid pathway 30 for reinfusion into the patient's vasculature.

In some embodiments, the two-way septum 66 may be configured to automatically open in response to the saline push in the distal direction 24. In some embodiments, the amount of pressure used to reinfuse blood through the second fluid pathway 30 may be less than the amount of pressure used to infuse fluid through the first fluid pathway 26. In some embodiments, the infusion portion 40 may be locked to prevent fluid flow therethrough. In some embodiments, this may contribute to the fluid-locked state such that the fluid may be naturally directed into the patient's vasculature rather than retrograding or moving in the proximal direction 38 back into the infusion portion 40.

In some embodiments, the adapter 12 may be disposed relatively close to the patient to reduce the amount of blood needed for blood withdrawal for a diagnostic procedure. Likewise, the adapter 12 positioned close to the patient may reduce an amount of saline or other suitable flushing fluid needed to reinfuse the blood back into the patient's vasculature.

In some embodiments, a first proximal port 68 may be aligned with a distal port 69 and the first fluid pathway 26 may be straight. In some embodiments, a second proximal port 71 may include a side port disposed between the first proximal port 68 and the distal port 69, which may facilitate removal of blood within the adapter 12 after blood withdraw. However, in some embodiments, the first fluid pathway 26 and the one-way septum 64 may be disposed second proximal port 71 and the distal port 69, and the second fluid pathway 30 and the two-way septum 66 may be disposed between the first proximal port 58 and the distal port 69.

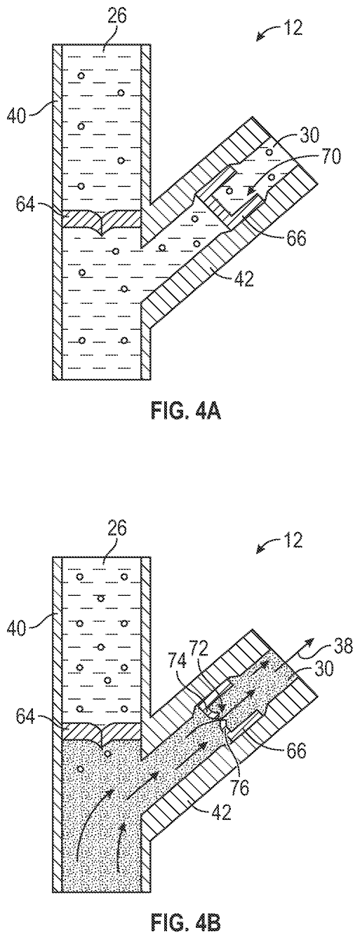

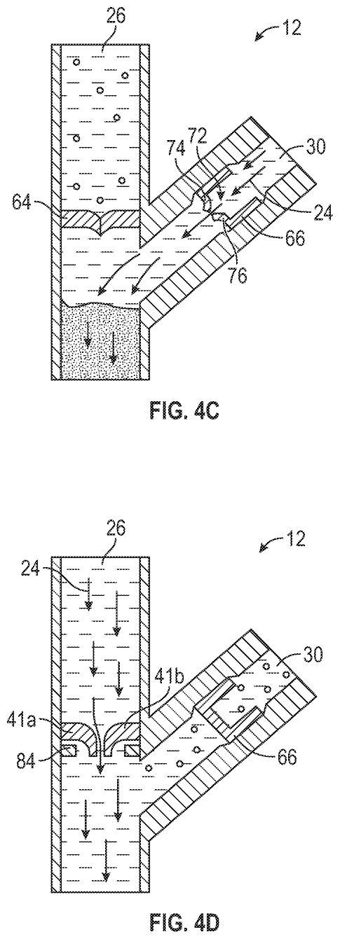

Referring now to FIG. 4A, in some embodiments, a method to facilitate fluid infusion and blood withdrawal in accordance with some embodiments may include priming the adapter 12, which may include the blood withdrawal portion 42 and the infusion portion 40. As illustrated, for example, both the one-way septum 64 and the two-way septum 66 may be closed such that no fluid is allowed to flow during the priming procedure.

FIG. 4A illustrates the infusion portion 40 and the blood withdrawal portion 42 each filled with priming fluid in accordance with some embodiments. In some embodiments, this priming fluid may be retained within the infusion portion 40 and the blood withdrawal portion 42 by the one-way septum 64 and the two-way septum 66, for example. In some embodiments, the priming fluid may be further retained within the adapter 12 by activating a pinch clamp (not illustrated) or another suitable clamp coupled to the third extension tube 48.

Referring now to FIG. 4B, in some embodiments, the method may include withdrawing blood from the patient's vasculature in the proximal direction 38 through the blood withdrawal portion 42. In some embodiments, as illustrated, the one-way septum 64 may be closed while the two-way septum 66 cracks open to allow blood withdrawal.

In some embodiments, the blood withdrawal portion 42 may include the second fluid pathway 30 to direct the blood into the blood collection reservoir 28. In some embodiments, negative pressure or a vacuum may be applied to the second fluid pathway 30 to cause the blood to flow in the proximal direction 38. In some embodiments, the negative pressure may cause the one-way septum 64 in the infusion portion 40 to create a fluid-tight seal.

In some embodiments, the two-way septum 66 may be configured to passively move between a closed position 70 and an open position 72. In some embodiments of the closed position 70, the two-way septum 66 may block the second fluid pathway 30, while in the open position 72, the two-way septum 66 may not block the second fluid pathway 30.

In some embodiments, the two-way septum 66 may include a first flexible portion 74 and a second flexible portion 76. In some embodiments, the first flexible portion 74 and the second flexible portion 76 may be configured to deform in the proximal direction 38 in response to the flow of blood in the proximal direction 38. In some embodiments, the first flexible portion 74 and the second flexible portion 76 may be further configured to deform in the distal direction 24 in response to a push of saline or another suitable fluid in the distal direction 24. Some embodiments may utilize the saline of fluid push to direct the flow of blood in the distal direction 24 to reinfuse blood into the patient's vasculature.

Referring now to FIG. 4C, in some embodiments, the method may include performing an analysis of the blood in the blood collection reservoir 28. Some embodiments may include pushing a second fluid in the distal direction 24 through the blood collection reservoir 28, the blood withdrawal portion 42 and/or the second fluid pathway 30 to thereby reinfuse the blood into the patient's vasculature following the analysis.

Referring now to FIG. 4D, in some embodiments, the method may enable a blood sample to be collected through a different fluid pathway from the fluid pathway that may be subsequently used to infuse a fluid. Importantly, this enables the infusion portion 40 of the adapter 12 to be pre-primed, and also enables collection of the blood sample while the infusion portion 40 remains primed.

During an infusion procedure, in some embodiments, the one-way septum 64 may be open while the two-way septum 66 may be closed. For example, the infusate or first fluid may be directed in the distal direction 24 through the infusion portion 40. In some embodiments, directing the infusate in the distal direction 24 through the infusion portion 40 may cause the one-way septum 64 disposed therein to automatically open. Some embodiments may thus open the first fluid pathway 26 such that the infusate may be infused through the infusion portion 40 and first fluid pathway 26 into the patient's vasculature.

Some embodiments of the one-way septum 64 may include a resilient element having a slit therethrough. In some embodiments, the slit may form a first resilient portion 41a and a second resilient portion 41b. In some embodiments, each of the first resilient portion 41a and the second resilient portion 41b may be configured to deflect in the distal direction 24. This may cause some embodiments of the one-way septum 64 to open in response to a flow of fluid in the distal direction 24, and to automatically provide a fluid-tight seal in response to cessation of the flow of fluid.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A catheter system, comprising:
a catheter hub comprising a distal end and a proximal end;
a catheter extending from the distal end of the catheter hub;
an adapter in fluid communication with the catheter hub and the catheter, wherein the adapter comprises a first proximal port, a second proximal port, and a distal port, wherein an infusion fluid pathway extends through the first proximal port and the distal port, wherein a blood withdrawal fluid pathway extends through the second proximal port and the distal port;
a one-way septum disposed in the first proximal port and configured to open to allow fluid flow in a distal direction at a pressure differential across the one-way septum, wherein the one-way septum is disposed within the infusion fluid pathway; and
a two-way septum disposed in the second proximal port, wherein the two-way septum is disposed within the blood withdrawal fluid pathway.

2. The catheter system of claim 1, further comprising an extension tube, wherein the extension tube comprises a distal end coupled to the catheter hub and a proximal end coupled to the distal port of the adapter.

3. The catheter system of claim 1, wherein the first proximal port is aligned with the distal port and the infusion fluid pathway is straight.

4. The catheter system of claim 3, wherein the second proximal port comprises a side port disposed between the first proximal port and the distal port.

5. The catheter system of claim 1, further comprising:
an infusion module, wherein the infusion module comprises a first extension tube configured to direct a fluid in a distal direction through the infusion fluid pathway for infusion into a patient's vasculature; and
a diagnostic module, wherein the diagnostic module comprises a second extension tube configured to direct blood in a proximal direction from the patient's vasculature through the blood withdrawal fluid pathway.

6. The catheter system of claim 5, wherein a distal end of the first extension tube is coupled to the first proximal port and a distal end of the second extension tube is coupled to the second proximal port.

7. The catheter system of claim 5, wherein the infusion module comprises at least one of a pump and a gravity-based device to direct the fluid in the distal direction through the infusion fluid pathway.

8. The catheter system of claim 5, wherein the diagnostic module is configured to apply a pressure differential across the two-way septum to open the two-way septum and direct blood in the proximal direction through the blood withdrawal fluid pathway.

9. The catheter system of claim 8, wherein the diagnostic module further comprises a pump to push a fluid in the distal direction through the blood withdrawal fluid pathway to reinfuse the blood into the patient's vasculature.

10. The catheter system of claim 9, wherein the diagnostic module further comprises at least one sensor to perform a diagnostic test on the blood.

11. The catheter system of claim 9, further comprising a saline flush device, wherein the pump is configured to reintroduce blood into the blood withdrawal pathway and distally through the catheter via the saline flush device.

12. The catheter system of claim 11, wherein the saline flush device is embedded in the diagnostic module.

13. The catheter system of claim 11, wherein the saline flush device stands alone with respect to the diagnostic module.

14. An adapter configured to couple to a catheter assembly, the adapter comprising:
a first proximal port;
a second proximal port; and
a distal port, wherein an infusion fluid pathway extends between the first proximal port and the distal port, wherein a blood withdrawal fluid pathway extends between the second proximal port and the distal port;
a one-way septum disposed in the first proximal port and configured to open to allow fluid flow in a distal direction at a pressure differential across the one-way septum, wherein the one-way septum is disposed within the infusion fluid pathway; and
a two-way septum disposed in the second proximal port, wherein the two-way septum is disposed within the blood withdrawal fluid pathway.

15. The adapter of claim 14, wherein the first proximal port is aligned with the distal port and the infusion fluid pathway is straight.

16. The adapter of claim 15, wherein the second proximal port comprises a side port disposed between the first proximal port and the distal port.

17. A method to facilitate fluid infusion and blood withdrawal, comprising:
priming an adapter, wherein the adapter comprises:
a first proximal port;
a second proximal port; and
a distal port, wherein an infusion fluid pathway extends between the first proximal port and the distal port, wherein a blood withdrawal fluid pathway extends between the second proximal port and the distal port;
a one-way septum disposed in the first proximal port and configured to open to allow fluid flow in a distal direction at a pressure differential across the one-way septum, wherein the one-way septum is disposed within the infusion fluid pathway; and
a two-way septum disposed in the second proximal port, wherein the two-way septum is disposed within the blood withdrawal fluid pathway;
directing a first fluid in a distal direction through the infusion pathway to infuse the first fluid into a patient's vasculature;
withdrawing blood from the patient's vasculature in a proximal direction through the blood withdrawal fluid pathway to direct the blood into a blood collection reservoir;
performing an analysis of the blood in the blood collection reservoir; and
after performing the analysis, pushing a second fluid in the distal direction through at least one of the blood collection reservoir and the blood withdrawal fluid pathway to reinfuse the blood into the patient's vasculature.

18. The method of claim 17, wherein directing the first fluid in the distal direction comprises utilizing a flow of the first fluid to open a one-way septum disposed within the first proximal port.

19. The method of claim 17, wherein in response to utilizing a flow of the first fluid to open the one-way septum, the two-way septum remains closed.

20. The method of claim 19, wherein in response to pushing the second fluid in the distal direction through at least one of the blood collection reservoir and the blood withdrawal fluid pathway to reinfuse the blood into the patient's vasculature, the one-way septum remains closed.
